(19) United States
(12) Patent Application Publication
(10) Pub. No.: US 2026/0055040 A1
(43) Pub. Date: Feb. 26, 2026

(54) POLYOL DEHYDRATION USING NOVEL SOLID ALUMINA COMPOSITION

(71) Applicant: BRASKEM AMERICA, INC., Philadelphia, PA (US)

(72) Inventors: Priya D. SRINIVASAN, Pittsburgh, PA (US); Dan Xie, Pittsburgh, PA (US); Jiahan Xie, Pittsburgh, PA (US)

(21) Appl. No.: 19/307,564

(22) Filed: Aug. 22, 2025

Related U.S. Application Data
(60) Provisional application No. 63/686,544, filed on Aug. 23, 2024.
(60) Provisional application No. 63/868,105, filed on Aug. 21, 2025.

(51) Int. Cl. C07C 29/60; B01J 6/00; B01J 21/04; B01J 35/30; B01J 35/77; B01J 37/08

(52) U.S. Cl. CPC C07C 29/60; B01J 6/001; B01J 21/04; B01J 35/30; B01J 35/77; B01J 37/08; B01J 2235/15

(57) ABSTRACT

The disclosure provides a process for a polyol dehydration, including: providing a feedstock containing a polyol, and dehydrating the polyol in the presence of a solid alumina composition as a catalyst or catalyst support for the dehydration reaction to obtain a dehydration product; wherein the solid alumina composition is prepared from a precursor composition comprising an alumina hydroxide (Al(OH)3), aluminum oxyhydroxide (AlO(OH)), or a mixture thereof.

### CROSS-REFERENCE TO RELATED APPLICATIONS

This application claims the benefit of priority under 35 U.S.C. § 119(e) to U.S. Provisional Patent Application No. 63/686,544, filed on Aug. 23, 2024, and U.S. Provisional Patent Application No. 63/868,105 filed on Aug. 21, 2025, both of which are herein incorporated by reference in their entirety.

### BACKGROUND OF THE INVENTION

Expanding a portfolio of biobased chemical production pathways is a key strategy in the polyolefin industry, to achieve sustainability goals set by the industry. From a technology perspective, developing robust catalyst systems that could either produce targeted biobased chemicals and improve their yields, or promote better energy efficiency to reduce carbon emissions in chemical processes, is an important competitive advantage. A goal therefore is to develop catalysts and catalyst systems with increased performance in dehydration pathways of alcohols and polyols, strengthening a technology portfolio in the field of biomass conversion to industrially important chemicals.

Polyol molecules can be readily derived from biobased feedstock, such as glycerol, ethylene glycol, propylene glycol, butanediol, etc. Dehydration is a common pathway to effectively remove oxygen and generate versatile C═C bond to final product. For example, the dehydration product of glycerol, acrolein, can then be further converted to valuable propanol, propylene, acrylic acid, etc.

However, polyol dehydration usually generates a large amount of byproducts, due to multiple functional hydroxyl groups on the reactant. Additionally, because polyol dehydration is an endothermic reaction, a high reaction temperature is usually needed, which in turn causes a polyol to be very reactive to form byproducts. Some recent efforts to scale up glycerol dehydration process were unsuccessful; the efforts stopped as the pilot plant scale, because of poor economics due to low yield of desired products (˜70% acrolein yield).

Thus, developing a reactive catalyst at a lower temperature with a high selectivity towards the target product, is of great significance to the success of this technology. This invention relates to the application of the novel solid alumina composition prepared from an acid-treated precursor composition to a polyol dehydration reaction with significantly improved performance (e.g., reactivity and selectivity to desired products).

The disclosure herein includes an exceptional performance of the novel solid alumina composition prepared from an acid-treated precursor composition for polyol dehydration application, as demonstrated by an exemplary reaction of 1,2-propanediol dehydration to produce propionaldehyde. Advantages of inventive processes include: the use of biobased, renewable feedstocks; increased energy efficiency and/or carbon emissions reductions; the use of a novel solid alumina composition prepared from an acid-treated precursor composition, which had a higher acid site concentration and provided a better performance compared to other alumina dehydration catalysts; a novel solid alumina composition prepared from an acid-treated precursor composition exhibited ˜50% improvement of space time yield compared to other alumina dehydration catalysts; and a tuning of the acid strength of the catalyst, inhibiting formation of dioxolane byproducts.

### BRIEF SUMMARY OF THE INVENTION

An embodiment of the present invention relates to a solid alumina composition prepared from an acid-treated precursor composition, said precursor composition comprising an alumina hydroxide (Al(OH)₃), aluminum oxyhydroxide (AlO(OH)), or a mixture thereof; wherein the solid alumina composition comprises η-alumina characterized by having an X-ray powder diffraction pattern comprising the peaks at 19.6±0.5 °2θ and 66.8±0.5 °2θ; and wherein the solid alumina composition has an acidity measured by an NH₃-TPD test, characterized by: (1) an acid site density ranging from about 200 to 800 μmol/g (such as from about 250 to about 800, from about 300 to about 800, or from about 400 to about 800 μmol/g); and/or (2) an acid strength distribution of: about 20%-70% weak acid sites (such as about 30-50%, about 30-40%, or about 35-40%), about 30%-80% medium acid sites (such as about 50-70%, about 60-70%, or about 60-65%), and about 0-20% strong acid sites (such as about 0-10%, about 0-5%, or about 0-2%).

Another embodiment of the present invention relates to a solid alumina composition above, which comprises η-alumina at a high purity, characterized by a ratio of a peak intensity at 19.6±0.5 °2θ, relative to a peak intensity at 66.8±0.5 °2θ, being about 3% or greater, preferably from about 3% to about 10%, or preferably from about 6% to about 10%.

Another embodiment of the present invention relates to a solid alumina composition above, which has an acidity measured by an NH₃-TPD test, characterized by: (1) an acid site density ranging from about 200 to 800 μmol.

Another embodiment of the present invention relates to a solid alumina composition above, which has an acidity measured by an NH₃-TPD test, characterized by: (2) an acid strength distribution of: about 20%-70% weak acid sites (such as about 30-50%, about 30-40%, or about 35-40%), about 30%-80% medium acid sites (such as about 50-70%, about 60-70%, or about 60-65%), and about 0-20% strong acid sites (such as about 0-10%, about 0-5%, or about 0-2%).

Another embodiment of the present invention relates to a solid alumina composition above, which comprises an acidity measured by an NH₃-TPD test, characterized by: (1) an acid site density ranging from about 250 to about 800, preferably from about 300 to about 800, or preferably from about 400 to about 800 μmol/g.

Another embodiment of the present invention relates to a solid alumina composition above, wherein the aluminum hydroxide comprises bayerite.

Another embodiment of the present invention relates to a solid alumina composition above, wherein the aluminum oxyhydroxide comprises boehmite (γ-AlO(OH)), pseudo-boehmite (e.g., finely crystalline boehmite), or a mixture thereof.

Another embodiment of the present invention relates to a solid alumina composition above, wherein the precursor composition comprises a mixture of bayerite and boehmite or pseudo-boehmite, optionally containing over 50 wt % (such as over 60 wt %, over 70 wt %, over 80 wt %, over 90 wt %, or over 95 wt %) bayerite.

Another embodiment of the present invention relates to a solid alumina composition above, wherein an acid to obtain the acid-treated precursor composition, comprises an acid selected from the group consisting of an inorganic acid, a halogen acid, an organic acid, and a mixture thereof.

Another embodiment of the present invention relates to a solid alumina composition above, wherein the acid comprises an inorganic acid selected from the group consisting of sulfuric acid, boric acid, nitric acid, and a mixture thereof.

Another embodiment of the present invention relates to a solid alumina composition above, wherein the acid comprises a halogen acid selected from the group consisting of hydrochloric acid, hydrobromic acid, and a mixture thereof.

Another embodiment of the present invention relates to a solid alumina composition above, wherein the acid comprises an organic acid selected from the group consisting of acetic acid, citric acid, malic acid, tartaric acid, propionic acid, butyric acid, valeric acid, lactic acid, hydracrylic acid, glyceric acid, levulinic acid, malonic acid, glutaric acid, succinic acid, pimelic acid, and a mixture thereof.

Another embodiment of the present invention relates to a solid alumina composition above, wherein the acid comprises an acid selected from the group consisting of nitric acid, acetic acid, citric acid, malic acid, tartaric acid, or a mixture thereof.

Another embodiment of the present invention relates to a process for preparing a solid alumina composition, the process comprising: treating a precursor composition comprising an alumina hydroxide (Al(OH)₃), aluminum oxyhydroxide (AlO(OH)), or a mixture thereof with an acid, to form an acid-treated precursor composition; dispersing the acid-treated precursor composition into a homogenous aqueous suspension of particles; drying the homogenous aqueous suspension of particles; and calcinating the dried particles, to form a solid alumina composition.

Another embodiment of the present invention relates to a process above, wherein the dispersing is carried out by sonication, stirring, shaking, or a combination thereof.

Another embodiment of the present invention relates to a process above, wherein adding a surfactant to the homogenous aqueous suspension of particles.

Another embodiment of the present invention relates to a process above, wherein the surfactant comprises a cationic surfactant, an anionic surfactant, or a combination thereof.

Another embodiment of the present invention relates to a process above, wherein the cationic surfactant comprises a salt of a quaternary ammonium cation selected from the group consisting of cetyltrimethylammonium, cetylpyridinium, polydially dimethyl ammonium, and a combination thereof, and wherein the anionic surfactant comprises a surfactant selected from the group consisting of an alkyl sulfate, an alkyl-ether sulfate, an alkylbenzene sulfonate, a perfluoroalkanesulfonate, an alkyl-aryl ether phosphate, an alkyl ether phosphate, sodium stearate, and a combination thereof.

Another embodiment of the present invention relates to a process above, wherein the non-ionic surfactant comprises at least one member selected from the group consisting of a fatty alcohol ethoxylate, an alkyl phenol ethoxylate, a fatty acid alkoxylate, an ethylene oxide/propylene oxide copolymer, and a poly(ethylene oxide)-based surfactant.

Another embodiment of the present invention relates to a process above, wherein the poly(ethylene oxide)-based surfactant comprises at least one of TERGITOL 15-S-5, TERGITOL 15-S-7, TERGITOL 15-S-9, and TERGITOL 15-S-12.

Another embodiment of the present invention relates to a process above, wherein the surfactant comprises cetyltrimethylammonium bromide, poly-diallyldimethylammonium chloride, or Tergitol 15-S-7.

Another embodiment of the present invention relates to a process above, wherein the drying is carried out at a temperature ranging from about 20° C. to about 200° C., preferably from about 50° C. to about 150° C., or preferably from about 90° C. to about 100° C.

Another embodiment of the present invention relates to a process above, further comprising, prior to calcinating: cooling the dried particles to an ambient temperature.

Another embodiment of the present invention relates to a process above, wherein the calcinating is carried out at a temperature ranging from about 400° C. to about 750° C., preferably from about 500° C. to about 650° C.

Another embodiment of the present invention relates to a process above, wherein the calcinating is carried out at a ramp rate of about 0.5° C./minute to about 2° C./minute (such as about 1° C./minute), from ambient temperature to reach a final temperature of from about 500° C. to about 650° C. (such as from about 600° C. to about 650° C.), and optionally maintaining at the final temperature for about 4-12 hours (e.g., about 6-10 hours, about 8-10 hours, or about 10 hours).

Another embodiment of the present invention relates to a process above, wherein the calcinating is carried out in a stepwise manner, comprising: a first calcinating from ambient temperature to a reach a first temperature ranging from about 400° C. to about 450° C. (such as about 400° C.), and maintaining at the first temperature for about 1-3 hours (e.g., about 2 hours); a second calcinating from the first temperature to a second temperature ranging from about 450° C. to about 550° C. (such as about 500° C.), and maintaining at the second temperature for about 1-3 hours (e.g., about 2 hours); and a third calcinating from the second temperature to the final temperature, and maintaining at the final temperature for about 4-8 hours (e.g., about 6 hours).

Another embodiment of the present invention relates to a solid alumina composition prepared according to any process herein, wherein the solid alumina composition comprises η-alumina characterized by having an X-ray power diffraction pattern comprising the peaks at 19.6.0±0.5 and 66.8±0.5 °2θ.

Another embodiment of the present invention relates to a solid alumina composition above, wherein the solid alumina composition is an acid type, optionally having an acidity measured by a NH₃-TPD test characterized by: (1) an acid site density ranging from about 200 to 800 μmol/g (such as from about 250 to about 800, from about 300 to about 800, or from about 400 to about 800 μmol/g); and/or (2) an acid strength distribution of: about 20%-70% weak acid sites (such as about 30-50%, about 30-40%, or about 35-40%), about 30%-80% medium acid sites (such as about 50-70%, about 60-70%, or about 60-65%), and about 0-20% strong acid sites (such as about 0-10%, about 0-5%, or about 0-2%).

Another embodiment of the present invention relates to a solid alumina composition above, wherein the solid alumina composition comprises η-alumina at a high purity, characterized by the ratio of the peak intensity at 20.0 (±0.2)° 2θ relative to the peak intensity at 67.0 (±0.2)° 2θ being about 3% or greater (such as ranging from about 3% to about 10%, or from about 6% to about 10%).

Another embodiment of the present invention relates to a process for a polyol dehydration, comprising: providing a feedstock containing a polyol, and dehydrating the polyol in the presence of a solid alumina composition as a catalyst or catalyst support for the dehydration reaction to obtain a dehydration product; wherein the solid alumina composition is prepared from a precursor composition comprising an alumina hydroxide (Al(OH)₃), aluminum oxyhydroxide (AlO(OH)), or a mixture thereof.

In any embodiment herein, the feedstock is a bio-based feedstock.

In any embodiment herein, the polyol is glycerol, ethane diol (i.e., ethylene glycol), a propane diol (such as 1,2-propanediol (i.e., propylene glycol), or 1,3-propanediol), or a butanediol (such as 1,2-butanediol, 1,3-butanediol, or 1,4-butanediol).

In any embodiment herein, the polyol is derived from sugar (e.g., maltitol, sorbitol, xylitol, erythritol, or isomalt), plant oil (e.g., castor oil or cottonseed oil), cellulose, or hemicellulose.

In any embodiment herein, the dehydration reaction has an improved reaction rate, characterized by a higher site time yield (i.e., a turnover rate) under a mild reaction conditions (e.g., at a reaction temperature ranging from about 200° C. to about 300° C., from about 220° C. to about 270° C., or at about 250° C.), wherein the site time yield is at least 10% (such as at least 15%, at least 20%, at least 25%, at least 30%, at least 35%, at least 40%, at least 45%, at least 50%, at least 60%, at least 70%, at least 80%, at least 90%, or at least 100%) higher, compared to the site time yield of a dehydration reaction carried out under the same conditions but in the presence of a commercial η- or γ-alumina as the catalyst or catalyst support.

In any embodiment herein, the site time yield of the dehydration reaction is at least 35% higher, at least 50% higher, or at least 100% higher, compared to the site time yield of a dehydration reaction carried out under the same conditions but in the presence of a commercial η- or γ-alumina as the catalyst or catalyst support.

In any embodiment herein, the polyol is 1,2-propanediol, and the dehydration reaction has a site time yield ranging from about 1.3 to about 1.7 kg_{reactant}kg⁻¹_{catalyst}hr⁻¹, under mild reaction conditions (e.g., at a reaction temperature ranging from about 200° C. to about 300° C., from about 220° C. to about 270° C., or at about 250° C.).

In any embodiment herein, the dehydration reaction has an improved selectivity toward a primary dehydration product, formed from eliminating a water molecule from the polyol, wherein the selectivity is improved by at least 50% (such as at least 60%, at least 70%, at least 80%, at least 90%, or at least 100%), compared to a dehydration reaction carried out under the same conditions but in the presence of a commercial η- or γ-alumina as the catalyst or catalyst support.

In any embodiment herein, the dehydration product contains a reduced amount of byproduct, wherein the byproduct is reduced by at least 50% (such as at least 60%), compared to a dehydration reaction carried out under the same conditions but in the presence of a commercial η- or γ-alumina as the catalyst or catalyst support.

In any embodiment herein, the dehydration product is formed from a dehydration process that involves eliminating a molecule other than water molecule, hydrogen transfer, and/or intra-molecular addition with secondary hydroxy group.

In any embodiment herein, the polyol is glycerol, and the primary dehydration product comprises at least one of 1,2-propanediol, acetol, and acrolein.

In any embodiment herein, the polyol is 1,2-propanediol, and the primary dehydration product comprises propionaldehyde.

In any embodiment herein, the dehydration product has: a selectivity toward propionaldehyde improved by about 50% (such as about 60%, about 70%, about 80%, or about 90%) or higher, and a reduced amount of 2-ethyl-4-methyle-1,3-dioxolane, which is reduced by about 50% (or about 60%) or more, compared to a dehydration reaction process carried out under the same conditions but in the presence of a commercial η- or γ-alumina as the catalyst or catalyst support.

In any embodiment herein, the dehydration reaction is carried out at a weight ratio of catalyst:polyol of 0.05-0.15 (e.g., about 0.1), a reaction temperature ranging from about 200° C. to about 300° C. (e.g., from about 220° C. to about 270° C., or at about 250° C.), and/or a stirring speed ranging from 250-350 rpm (e.g., about 300 rpm).

In any embodiment herein, the dehydration product contains: greater than about 60 mol % propionaldehyde, no more than about 11 mol % 2-ethyl-4-methyle-1,3-dioxolane, and/or no more than about 15 mol % propanol.

In any embodiment herein, the solid alumina composition comprises η-alumina characterized by having an X-ray powder diffraction pattern comprising the peaks at 19.6±0.5 °2θ (e.g., 19.6±0.2 °2θ) and 66.8±0.5 °2θ (e.g., 66.8±0.2 °2θ); and the solid alumina composition has an acidity measured by an NH₃-TPD test, characterized by: an acid site density ranging from about 200 to 800 μmol/g (such as from about 250 to about 800, from about 300 to about 800, or from about 400 to about 800 μmol/g); and/or an acid strength distribution of:
a. about 20%-70% weak acid sites (such as about 30-50%, about 30-40%, or about 35-40%),
b. about 30%-80% medium acid sites (such as about 50-70%, about 60-70%, or about 60-65%), and
c. about 0-20% strong acid sites (such as about 0-10%, about 0-5%, or about 0-2%).

In any embodiment herein, the solid alumina composition comprises η-alumina at a high purity, characterized by a ratio of a peak intensity at 19.6±0.5 °2θ (e.g., 19.6±0.2 °2θ), relative to a peak intensity at 66.8±0.5 °2θ (e.g., 66.8±0.2 °2θ), being about 3% or greater, preferably from about 3% to about 10%, or preferably from about 6% to about 10%.

In any embodiment herein, aluminum hydroxide comprises bayerite.

In any embodiment herein, aluminum oxyhydroxide comprises boehmite (γ-AlO(OH)), pseudo-boehmite (e.g., finely crystalline boehmite), or a mixture thereof.

In any embodiment herein, the precursor composition comprises a mixture of bayerite and boehmite or pseudo-boehmite, optionally containing over 50 wt % (such as over 60 wt %, over 70 wt %, over 80 wt %, over 90 wt %, or over 95 wt %) bayerite.

In any embodiment herein, an acid to obtain an acid-treated precursor composition comprises an acid selected from the group consisting of an inorganic acid (e.g., sulfuric acid, boric acid, nitric acid, or a mixture thereof), a halogen acid (e.g., hydrochloric acid, hydrobromic acid, or a mixture thereof), an organic acid (e.g., acetic acid, citric acid, malic acid, tartaric acid, propionic acid, butyric acid, valeric acid, lactic acid, hydracrylic acid, glyceric acid, levulinic acid, malonic acid, glutaric acid, succinic acid, pimelic acid, or a mixture thereof), and a mixture thereof.

In any embodiment herein, the acid comprises an acid selected from the group consisting of nitric acid, acetic acid, citric acid, malic acid, tartaric acid, and a mixture thereof.

In any embodiment herein, the solid alumina composition is prepared by a process comprising: treating a precursor composition comprising an alumina hydroxide (Al(OH)₃), aluminum oxyhydroxide (AlO(OH)), or a mixture thereof with an acid, to form an acid-treated precursor composition; dispersing the acid-treated precursor composition into a homogenous aqueous suspension of particles; drying the homogenous aqueous suspension of particles (e.g., at a temperature ranging from about 20° C. to about 200° C., preferably from about 50° C. to about 150° C., or preferably from about 90° C. to about 100° C.); and calcinating the dried particles (e.g., at a temperature ranging from about 400° C. to about 750° C., preferably from about 500° C. to about 650° C.), to form a solid alumina composition.

In any embodiment herein, the dispersing is carried out by sonication, stirring, shaking, or a combination thereof.

In any embodiment herein, the process for preparing the solid alumina composition further comprises: adding a surfactant (e.g., a cationic surfactant, an anionic surfactant, or a combination thereof) to the homogenous aqueous suspension of particles; and/or prior to calcinating, cooling the dried particles to an ambient temperature.

In any embodiment herein, the calcinating is carried out at a ramp rate of about 0.5° C./minute to about 2° C./minute (such as about 1° C./minute), from ambient temperature to reach a final temperature of from about 500° C. to about 650° C. (such as from about 600° C. to about 650° C.), and optionally maintaining at the final temperature for about 4-12 hours (e.g., about 6-10 hours, about 8-10 hours, or about 10 hours).

In any embodiment herein, the calcinating is carried out in a stepwise manner, comprising: a first calcinating from ambient temperature to a reach a first temperature ranging from about 400° C. to about 450° C. (such as about 400° C.), and maintaining at the first temperature for about 1-3 hours (e.g., about 2 hours); a second calcinating from the first temperature to a second temperature ranging from about 450° C. to about 550° C. (such as about 500° C.), and maintaining at the second temperature for about 1-3 hours (e.g., about 2 hours); and a third calcinating from the second temperature to the final temperature, and maintaining at the final temperature for about 4-8 hours (e.g., about 6 hours).

### BRIEF DESCRIPTION OF SEVERAL VIEWS OF THE DRAWING

FIG. 1 shows a XRPD patterns for γ-alumina and η-alumina.

FIG. 2 shows reaction products of 1,2-propanediol dehydration.

FIG. 3 shows reaction products of glycerol dehydration.

### DETAILED DESCRIPTION OF THE INVENTION

The indefinite articles “a” and “an” generally mean “at least one” in the sense of “a” or “an”. The indefinite article “a” can also mean “any.” Those skilled in the art will understand that the indefinite article “a” does not necessarily mean the indefinite article “a” but rather the indefinite article “a” in the sense of “1”, and that in one embodiment the indefinite article “a” also includes the indefinite article “a” (1). Furthermore, the indefinite article can mean “one” or “at least one.”

### A Solid Alumina Composition

An embodiment of the present invention relates to a solid alumina composition prepared from an acid-treated precursor composition. The precursor composition comprises, consists of, or has, an alumina hydroxide (Al(OH)₃), aluminum oxyhydroxide (AlO(OH)), or a mixture thereof. The mixture can be any two or all of these components. The solid alumina composition can comprise, or consist of, η-alumina characterized by having an X-ray powder diffraction pattern comprising peaks at 19.6±0.5 °2θ and 66.8±0.5 °2θ.

The solid alumina composition can comprise, or have, an acidity measured by an NH₃-TPD test, characterized by: (1) an acid site density ranging from about 200 to 800 μmol/g (such as from about 250 to about 800, from about 300 to about 800, or from about 400 to about 800 μmol/g); and/or (2) an acid strength distribution of: about 20%-70% weak acid sites (such as about 30-50%, about 30-40%, or about 35-40%), about 30%-80% medium acid sites (such as about 50-70%, about 60-70%, or about 60-65%), and about 0-20% strong acid sites (such as about 0-10%, about 0-5%, or about 0-2%). The NH₃-TPD is described below, under

### Characterization Tests

A solid alumina composition herein can comprise or have an acidity measured by an NH₃-TPD test, characterized by: (1) an acid site density ranging from about 200 to 800 μmol. The density can range from about 250 to about 800, or from about 300 to about 800, or from about 400 to about 800 μmol/g. All values between these minima and maxima are included in this description.

A solid alumina composition herein can comprise or have an acidity measured by an NH₃-TPD test, characterized by: (2) an acid strength distribution of: about 20%-70% weak acid sites, about 30%-80% medium acid sites, and about 0-20% strong acid sites. Regarding the weak acid sites, preferably, the range can be about 30-50%, or about 30-40%, or about 35-40%, relative to all acid sites in the solid alumina composition. All values between these minima and maxima are included in this description. Regarding the medium acid sites, preferably, the range can be about 50-70%, or about 60-70%, or about 60-65%), relative to all acid sites in the solid alumina composition. All values between these minima and maxima are included in this description. Regarding the strong acid sites, preferably, the range can be about 0-10%, or about 0-5%, or about 0-2%, relative to all acid sites in the solid alumina composition.

In an embodiment of a solid alumina composition herein, the solid alumina composition can comprise, or consist of, a η-alumina at a high purity. The high purity can be characterized by a ratio of a peak intensity at 19.6±0.5 °2θ, relative to a peak intensity at 66.8±0.5 °2θ, of the η-alumina, being about 3% or greater. Preferably, this ratio can be from about 3% to about 10%, or preferably from about 6% to about 10%. All values between these minima and maxima are included in this description.

In an embodiment, the aluminum hydroxide of the solid alumina composition, can comprise, or consist of, bayerite. Alternatively, or in addition to bayerite, the aluminum oxyhydroxide can comprise, or consist of, boehmite (γ-AlO(OH)), pseudo-boehmite (e.g., finely crystalline boehmite), or a mixture thereof.

In an embodiment, the precursor composition can comprise, or consist of, a mixture of bayerite and boehmite or pseudo-boehmite, optionally containing over 50 wt %, preferably over 60 wt %, or over 70 wt %, or over 80 wt %, or over 90 wt %, or over 95 wt %, bayerite. All values between these minima and maxima are included in this description.

In an embodiment, the acid-treated precursor composition can be obtained by treating a precursor composition discussed herein with an acid. In embodiments, the acid can comprise, or consist of, an acid selected from the group consisting of an inorganic acid, a halogen acid, an organic acid, and a mixture thereof. In embodiments, the acid comprises an inorganic acid selected from the group consisting of sulfuric acid, boric acid, nitric acid, and a mixture thereof. In embodiments, the acid comprises a halogen acid selected from the group consisting of hydrochloric acid, hydrobromic acid, and a mixture thereof. These acids can be by themselves or in a mixture of any other acid listed herein.

In an embodiment of a solid alumina composition herein, the acid can comprise, or consist of, an organic acid selected from the group consisting of acetic acid, citric acid, malic acid, tartaric acid, propionic acid, butyric acid, valeric acid, lactic acid, hydracrylic acid, glyceric acid, levulinic acid, malonic acid, glutaric acid, succinic acid, pimelic acid, and a mixture thereof. These acids can be by themselves or in a mixture of any other acid listed herein.

In an embodiment of a solid alumina composition herein, the acid can comprise, or consist of, an acid selected from the group consisting of nitric acid, acetic acid, citric acid, malic acid, tartaric acid, or a mixture thereof. These acids can be by themselves or in a mixture of any other acid listed herein.

### Process for Preparing a Solid Alumina Composition

Another embodiment of the present invention relates to a process for preparing a solid alumina composition, the process comprising: treating a precursor composition comprising an alumina hydroxide (Al(OH)₃), aluminum oxyhydroxide (AlO(OH)), or a mixture thereof with an acid, to form an acid-treated precursor composition; dispersing the acid-treated precursor composition into a homogenous aqueous suspension of particles; drying the homogenous aqueous suspension of particles; and calcinating the dried particles, to form a solid alumina composition.

In an embodiment of a process of preparing a solid alumina composition herein, the dispersing can be, or is, carried out by sonication, stirring, shaking, or a combination thereof.

In an embodiment of a process of preparing a solid alumina composition herein, the process can further comprise adding a surfactant to the homogenous aqueous suspension of particles. In embodiments, the surfactant can comprise, or consist of, a cationic surfactant, an anionic surfactant, or a combination thereof. In other embodiments, separate from or in addition to the cationic surfactant and/or anionic surfactant, the surfactant can comprise, or consist of, a non-ionic surfactant.

The cationic surfactant can comprise, or consist of, a salt of a quaternary ammonium cation selected from the group consisting of cetyltrimethylammonium, cetylpyridinium, polydially dimethyl ammonium, and a combination thereof. The anionic surfactant can comprise, or consist of, a surfactant selected from the group consisting of an alkyl sulfate, an alkyl-ether sulfate, an alkylbenzene sulfonate, a perfluoroalkanesulfonate, an alkyl-aryl ether phosphate, an alkyl ether phosphate, sodium stearate, and a combination thereof. The non-ionic surfactant can comprise, or consist of, at least one member selected from the group consisting of a fatty alcohol ethoxylate, an alkyl phenol ethoxylate, a fatty acid alkoxylate, an ethylene oxide/propylene oxide copolymer, and a poly(ethylene oxide)-based surfactant.

In embodiments, the poly(ethylene oxide)-based surfactant can comprise, or consist of, at least one of TERGITOL 15-S-5, TERGITOL 15-S-7, TERGITOL 15-S-9, and TERGITOL 15-S-12. In embodiments, the surfactant can comprise, or consist of, cetyltrimethylammonium bromide, poly-diallyldimethylammonium chloride, or TERGITOL 15-S-7.

In embodiments, the drying is carried out at a temperature ranging from about 20° C. to about 200° C., preferably from about 50° C. to about 150° C., or preferably from about 90° C. to about 100° C. In embodiments, the process can further comprise, prior to calcinating: cooling the dried particles to an ambient temperature.

In embodiments, the calcinating is carried out at a temperature ranging from about 400° C. to about 750° C., preferably from about 500° C. to about 650° C. The calcinating can be carried out at a ramp rate of about 0.5° C./minute to about 2° C./minute (such as about 1° C./minute), from ambient temperature to reach a final temperature of from about 500° C. to about 650° C. (such as from about 600° C. to about 650° C.), and optionally maintaining at the final temperature for about 4-12 hours (e.g., about 6-10 hours, about 8-10 hours, or about 10 hours).

In embodiments, the calcinating is carried out in a stepwise manner, that can comprise: a first calcinating from ambient temperature to a reach a first temperature ranging from about 400° C. to about 450° C. (such as about 400° C.), and maintaining at the first temperature for about 1-3 hours (e.g., about 2 hours); a second calcinating from the first temperature to a second temperature ranging from about 450° C. to about 550° C. (such as about 500° C.), and maintaining at the second temperature for about 1-3 hours (e.g., about 2 hours); and a third calcinating from the second temperature to the final temperature, and maintaining at the final temperature for about 4-8 hours (e.g., about 6 hours).

### Solid Alumina Composition Made by a Process

Another embodiment of the present invention relates to a solid alumina composition prepared according to any process herein, wherein the solid alumina composition comprises η-alumina characterized by having an X-ray power diffraction pattern comprising the peaks at 19.6.0±0.5 and 66.8±0.5 °2θ. The solid alumina composition can be an acid type, optionally having an acidity measured by a NH₃-TPD test characterized by: (1) an acid site density ranging from about 200 to 800 μmol/g (such as from about 250 to about 800, from about 300 to about 800, or from about 400 to about 800 μmol/g); and/or (2) an acid strength distribution of: about 20%-70% weak acid sites (such as about 30-50%, about 30-40%, or about 35-40%), about 30%-80% medium acid sites (such as about 50-70%, about 60-70%, or about 60-65%), and about 0-20% strong acid sites (such as about 0-10%, about 0-5%, or about 0-2%).

In another embodiment, in the solid alumina composition herein, the solid alumina composition can comprise η-alumina at a high purity, characterized by the ratio of the peak intensity at 20.0 (±0.2)° 2θ relative to the peak intensity at 67.0 (±0.2)° 2θ being about 3% or greater (such as ranging from about 3% to about 10%, or from about 6% to about 10%).

### Process of Polyol Dehydration

Another embodiment of the present invention relates to a process for a polyol dehydration, comprising: providing a feedstock containing a polyol, and dehydrating the polyol in the presence of a solid alumina composition as a catalyst or catalyst support for the dehydration reaction to obtain a dehydration product; wherein the solid alumina composition is prepared from a precursor composition comprising an alumina hydroxide (Al(OH)₃), aluminum oxyhydroxide (AlO(OH)), or a mixture thereof.

In any embodiment herein, the feedstock is a bio-based feedstock.

In any embodiment herein, the polyol is glycerol, ethane diol (i.e., ethylene glycol), a propane diol (such as 1,2-propanediol (i.e., propylene glycol), or 1,3-propanediol), or a butanediol (such as 1,2-butanediol, 1,3-butanediol, or 1,4-butanediol).

In any embodiment herein, the polyol is derived from sugar (e.g., maltitol, sorbitol, xylitol, erythritol, or isomalt), plant oil (e.g., castor oil or cottonseed oil), cellulose, or hemicellulose.

In any embodiment herein, the dehydration reaction has an improved reaction rate, characterized by a higher site time yield (i.e., a turnover rate) under a mild reaction conditions (e.g., at a reaction temperature ranging from about 200° C. to about 300° C., from about 220° C. to about 270° C., or at about 250° C.), wherein the site time yield is at least 10% (such as at least 15%, at least 20%, at least 25%, at least 30%, at least 35%, at least 40%, at least 45%, at least 50%, at least 60%, at least 70%, at least 80%, at least 90%, or at least 100%) higher, compared to the site time yield of a dehydration reaction carried out under the same conditions but in the presence of a commercial η- or γ-alumina as the catalyst or catalyst support.

In any embodiment herein, the site time yield of the dehydration reaction is at least 35% higher, at least 50% higher, or at least 100% higher, compared to the site time yield of a dehydration reaction carried out under the same conditions but in the presence of a commercial η- or γ-alumina as the catalyst or catalyst support.

In any embodiment herein, the polyol is 1,2-propanediol, and the dehydration reaction has a site time yield ranging from about 1.3 to about 1.7 kg_{reactant}kg⁻¹_{catalyst}hr⁻¹, under mild reaction conditions (e.g., at a reaction temperature ranging from about 200° C. to about 300° C., from about 220° C. to about 270° C., or at about 250° C.).

In any embodiment herein, the dehydration reaction has an improved selectivity toward a primary dehydration product, formed from eliminating a water molecule from the polyol, wherein the selectivity is improved by at least 50% (such as at least 60%, at least 70%, at least 80%, at least 90%, or at least 100%), compared to a dehydration reaction carried out under the same conditions but in the presence of a commercial η- or γ-alumina as the catalyst or catalyst support.

In any embodiment herein, the dehydration product contains a reduced amount of byproduct, wherein the byproduct is reduced by at least 50% (such as at least 60%), compared to a dehydration reaction carried out under the same conditions but in the presence of a commercial η- or γ-alumina as the catalyst or catalyst support.

In any embodiment herein, the dehydration product is formed from a dehydration process that involves eliminating a molecule other than water molecule, hydrogen transfer, and/or intra-molecular addition with secondary hydroxy group.

In any embodiment herein, the polyol is glycerol, and the primary dehydration product comprises at least one of 1,2-propanediol, acetol, and acrolein.

In any embodiment herein, the polyol is 1,2-propanediol, and the primary dehydration product comprises propionaldehyde.

In any embodiment herein, the dehydration product has: a selectivity toward propionaldehyde improved by about 50% (such as about 60%, about 70%, about 80%, or about 90%) or higher, and a reduced amount of 2-ethyl-4-methyle-1,3-dioxolane, which is reduced by about 50% (or about 60%) or more, compared to a dehydration reaction process carried out under the same conditions but in the presence of a commercial η- or γ-alumina as the catalyst or catalyst support.

In any embodiment herein, the dehydration reaction is carried out at a weight ratio of catalyst:polyol of 0.05-0.15 (e.g., about 0.1), a reaction temperature ranging from about 200° C. to about 300° C. (e.g., from about 220° C. to about 270° C., or at about 250° C.), and/or a stirring speed ranging from 250-350 rpm (e.g., about 300 rpm).

In any embodiment herein, the dehydration product contains: greater than about 60 mol % propionaldehyde, no more than about 11 mol % 2-ethyl-4-methyle-1,3-dioxolane, and/or no more than about 15 mol % propanol.

In any embodiment herein, the solid alumina composition comprises η-alumina characterized by having an X-ray powder diffraction pattern comprising the peaks at 19.6±0.5 °2θ (e.g., 19.6±0.2 °2θ) and 66.8±0.5 °2θ (e.g., 66.8±0.2 °2θ); and the solid alumina composition has an acidity measured by an NH₃-TPD test, characterized by: an acid site density ranging from about 200 to 800 μmol/g (such as from about 250 to about 800, from about 300 to about 800, or from about 400 to about 800 μmol/g); and/or an acid strength distribution of:
a. about 20%-70% weak acid sites (such as about 30-50%, about 30-40%, or about 35-40%),
b. about 30%-80% medium acid sites (such as about 50-70%, about 60-70%, or about 60-65%), and
c. about 0-20% strong acid sites (such as about 0-10%, about 0-5%, or about 0-2%).

In any embodiment herein, the solid alumina composition comprises η-alumina at a high purity, characterized by a ratio of a peak intensity at 19.6±0.5 °2θ (e.g., 19.6±0.2 °2θ), relative to a peak intensity at 66.8±0.5 °2θ (e.g., 66.8±0.2 °2θ), being about 3% or greater, preferably from about 3% to about 10%, or preferably from about 6% to about 10%.

In any embodiment herein, aluminum hydroxide comprises bayerite.

In any embodiment herein, aluminum oxyhydroxide comprises boehmite (γ-AlO(OH)), pseudo-boehmite (e.g., finely crystalline boehmite), or a mixture thereof.

In any embodiment herein, the precursor composition comprises a mixture of bayerite and boehmite or pseudo-boehmite, optionally containing over 50 wt % (such as over 60 wt %, over 70 wt %, over 80 wt %, over 90 wt %, or over 95 wt %) bayerite.

In any embodiment herein, an acid to obtain an acid-treated precursor composition comprises an acid selected from the group consisting of an inorganic acid (e.g., sulfuric acid, boric acid, nitric acid, or a mixture thereof), a halogen acid (e.g., hydrochloric acid, hydrobromic acid, or a mixture thereof), an organic acid (e.g., acetic acid, citric acid, malic acid, tartaric acid, propionic acid, butyric acid, valeric acid, lactic acid, hydracrylic acid, glyceric acid, levulinic acid, malonic acid, glutaric acid, succinic acid, pimelic acid, or a mixture thereof), and a mixture thereof.

In any embodiment herein, the acid comprises an acid selected from the group consisting of nitric acid, acetic acid, citric acid, malic acid, tartaric acid, and a mixture thereof.

In any embodiment herein, the solid alumina composition is prepared by a process comprising: treating a precursor composition comprising an alumina hydroxide (Al(OH)₃), aluminum oxyhydroxide (AlO(OH)), or a mixture thereof with an acid, to form an acid-treated precursor composition; dispersing the acid-treated precursor composition into a homogenous aqueous suspension of particles; drying the homogenous aqueous suspension of particles (e.g., at a temperature ranging from about 20° C. to about 200° C., preferably from about 50° C. to about 150° C., or preferably from about 90° C. to about 100° C.); and calcinating the dried particles (e.g., at a temperature ranging from about 400° C. to about 750° C., preferably from about 500° C. to about 650° C.), to form a solid alumina composition.

In any embodiment herein, the dispersing is carried out by sonication, stirring, shaking, or a combination thereof.

In any embodiment herein, the process for preparing the solid alumina composition further comprises: adding a surfactant (e.g., a cationic surfactant, an anionic surfactant, or a combination thereof) to the homogenous aqueous suspension of particles; and/or prior to calcinating, cooling the dried particles to an ambient temperature.

In any embodiment herein, the calcinating is carried out at a ramp rate of about 0.5° C./minute to about 2° C./minute (such as about 1° C./minute), from ambient temperature to reach a final temperature of from about 500° C. to about 650° C. (such as from about 600° C. to about 650° C.), and optionally maintaining at the final temperature for about 4-12 hours (e.g., about 6-10 hours, about 8-10 hours, or about 10 hours).

In any embodiment herein, the calcinating is carried out in a stepwise manner, comprising: a first calcinating from ambient temperature to a reach a first temperature ranging from about 400° C. to about 450° C. (such as about 400° C.), and maintaining at the first temperature for about 1-3 hours (e.g., about 2 hours); a second calcinating from the first temperature to a second temperature ranging from about 450° C. to about 550° C. (such as about 500° C.), and maintaining at the second temperature for about 1-3 hours (e.g., about 2 hours); and a third calcinating from the second temperature to the final temperature, and maintaining at the final temperature for about 4-8 hours (e.g., about 6 hours).

### Characterization Tests

NH₃-TPD test: The acidity (i.e. acid sites) of the solid alumina composition was characterized by ammonia temperature programmed desorption (NH₃-TPD) test, using AutoChem II instrument. The produced solid alumina composition was pretreated with 10 ccm Ar at 400° C. for 2 hours, and the sample was exposed to the flow of NH₃ in Ar for 1 hour at 50° C. After NH₃ exposure, the sample was purged under the flow of Ar for 1 hour to removed weakly adsorbed NH₃, followed by heating under the flow of Ar (10 ccm Ar) to 625° C. at 10° C./min.

The NH₃-TPD profile was measured using a mass spectrometer, to quantify the sample acid site concentration. The “weak” acid sites were defined as the acid sites having a desorption temperature lower than 200° C.; the “medium” acid sites were defined as the acid sites having a desorption temperature range from 200 to 450° C., and the “strong” acid sites were defined as the acid sites having a desorption temperature higher than 450° C. A detailed discussion of the NH₃-TPD experiment and procedure can be found in: J. L. Falconer, J. A. Schwarz, Temperature-programmed desorption and reaction: applications to supported catalysts, Catal. Rev. 25 (June 2) (1983) 141-227.

X-ray Powder Diffraction (XRPD): XRPD patterns were collected on a diffractometer (such as a PANalytical X'Pert PRO or equivalent) using an incident beam of Cu Kα radiation (45 kV, 40 mA), θ-θ goniometer, focusing mirror, divergence slit (½″), soller slits at both incident and divergent beam (4 mm) under ambient conditions. The data collection range was 3-35° 2θ with a continuous scan speed of 0.2° s⁻¹.

The term “peak” refers to a signal that represents a reflection in the x-ray powder diffraction pattern. The peaks characterizing η-alumina in the x-ray powder diffraction pattern should be clear and distinct with low or minimum background noise.

### EXAMPLES

### Example 1

5.2 g of Bayerite hydroxide powder (PURAL BT from Sasol) was added to 50 mL of a 0.4M nitric acid solution under vigorous stirring. The suspension was sonicated for at least 10 minutes to achieve a homogeneous dispersion of the aluminum hydroxide particles. Then, 0.2 g of a 20 wt % poly-diallyldimethylammonium chloride solution was added to the gel under vigorous stirring. The mixture was then dried in open container at 95° C. for 34 hours and cooled down to room temperature. The final alumina product, BCM-1, was obtained by further calcining the dried powder sample under air flow from room temperature to 400° C. with a ramp rate of 1° C./minute and kept for 2 hours, then to 500° C. with the same ramp rate and kept for 2 hours, and to 600° C. with the same ramp rate and kept for 6 hours.

Acid site measurements according to the NH₃-TPD Characterization Tests described herein were performed on BCM-1. The results are shown in Table 1.

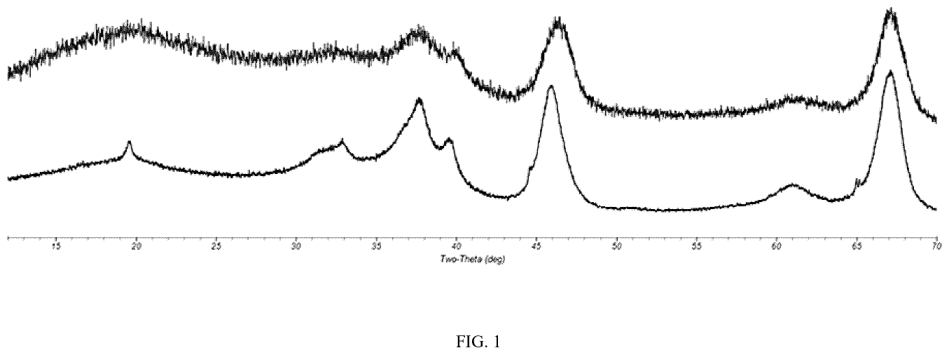

FIG. 1 shows XRPD patterns for γ-alumina and η-alumina of BCM-1. The top pattern is for the γ-alumina fraction of BCM-1, and the bottom pattern is for the η-alumina fraction of BCM-1. The XRPD patterns for BCM-1 were obtained via the X-ray Powder Diffraction method of the Characterization Tests described herein.

### Example 2

7.8 g of Bayerite hydroxide powder (PURAL BT from Sasol) was added to 80 g of deionized water and 10 mL of a 0.1M nitric acid solution under vigorous stirring. Then, 19.21 g of citric acid was slowly added to the solution under vigorous stirring at 80° C. The gel was stirred for at least 2 hours to ensure a homogeneous dispersion of the aluminum hydroxide particles, before 2.6 g of TERGITOL 15-S-7 was added. The mixture was then dried in open container at 95° C. for 60 hours and cooled down to room temperature. The final alumina product was obtained by further calcining the dried powder sample under air flow from room temperature to 400° C. with a ramp rate of 1° C./minute and kept for 2 hours, then to 500° C. with the same ramp rate and kept for 2 hours, and to 600° C. with the same ramp rate and kept for 6 hours.

### Example 3

6.0 g of Bayerite hydroxide powder (PURAL BT from Sasol) was added to 60 mL of a 0.2M nitric acid solution under vigorous stirring. The suspension was sonicated for at least 10 minutes to achieve a homogeneous dispersion of the aluminum hydroxide particles. Then, 0.6 g of a 20 wt % poly-diallyldimethylammonium chloride solution was added to the gel under vigorous stirring. The mixture was then dried in open container at 95° C. for 48 hours and cooled down to room temperature. The dried sample was further calcined under air flow from room temperature to 650° C. with a ramp rate of 1° C./minute and kept for 10 hours to obtain a final alumina product.

TABLE 1

| Catalyst | Weak acid sites | Medium acid sites | Strong acid sties |
|---|---|---|---|
| BCM-1 from Example 1 | 157 | 250 | 0 |
| Comparative γ-alumina | 137 | 46 | 11 |
| (CatalOX Sba-200 from | | | |
| Sasol) | | | |

### Example 4

### Catalyst Synthesis:

The catalyst used herein is an eta (h) alumina-based material. It is solid alumina composition that may be prepared from an acid-treated precursor composition synthesized using alumina hydroxide (preferably Bayerite), or a mixture of alumina hydroxide and alumina oxyhydroxide with the former being the main component (preferably a mixture of Bayerite and Boehmite/Pseudo-boehmite with Bayerite being the main component), as the precursor material.

The synthesis process may involve (1) precursor material acid treatment, (2) dispersion, (3) surfactant enhancement, (4) drying, and (5) calcination.

An exemplary procedure for synthesizing an exemplary solid alumina composition suitable for polyol dehydration is shown below.

5.2 g of Bayerite hydroxide powder (PURAL BT from Sasol) was added to 50 mL of a 0.4M nitric acid solution under vigorous stirring. The suspension was sonicated for at least 10 minutes to achieve a homogeneous dispersion of the aluminum hydroxide particles. Then, 0.2 g of a 20 wt % poly-diallyldimethylammonium chloride solution was added to the gel under vigorous stirring. The mixture was then dried in open container at 95° C. for 34 hours and cooled down to room temperature. The final product was obtained by further calcining the dried powder sample under air flow from room temperature to 400° C. with a ramp rate of 1° C./minute and kept for 2 hours, then to 500° C. with the same ramp rate and kept for 2 hours, and to 600° C. with the same ramp rate and kept for 6 hours.

Polyol dehydration reactions: Here, 1,2 propanediol and glycerol were used as an exemplary polyol for polyol dehydration.

### 1,2-Propanediol Dehydration:

Catalyst testing was performed in a 45 mL batch reactor (Parr Instruments 5000 series multiple reaction system). In a typical experiment, the reactor was loaded with the catalyst, the solid alumina composition synthesized above (0.15 g), and 1,2-propanediol (1.5 g) dissolved in a solvent consisting of 6 mL of water. The reactor was sealed, purged with 1000 psi of Helium three times, at ambient temperature. The reactor was then heated to 250° C. and once the desired temperature was attained (usually within 25 minutes), the stirring speed was set to 300 rpm, signifying the start of reaction. Following the fixed-time batch run for 2 hours, the heating and stirring were simultaneously stopped. The reactor was allowed to cool to ambient temperature in air, after which product samples were taken out and a known amount of internal standard (1,4-butanediol) was added. The product samples were firstly centrifuged using VWR® fixed angle centrifuge at 4000 RPM for 10 minutes to separate the catalyst particles from the liquid product samples. Then the centrifuged liquid samples were filtered through a polyethersulfone syringe membrane with 0.2 μm pore size to further separate the fine particles remaining in the liquid samples. These samples were analyzed by an Agilent Technologies 8890 gas Chromatograph equipped with a flame ionization detector (FID). All the samples were injected automatically by an Agilent Technologies 7693 Series auto-injector with a 10 μL syringe. A DB-WAX Ultra Inert capillary column (30 m×0.25 mm I.D.×0.50 μm film thickness) was used for separation of different species. Three experiments were performed under identical experiment conditions, to calculate the average and standard deviation. The carbon mass balance was closed at about 85% for this test.

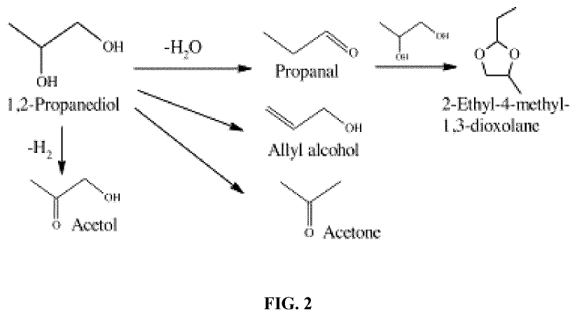

The reaction path is shown in FIG. 2. Dehydration of 1,2-propanediol proceeds via E1 elimination by protonation of the hydroxyl group and the subsequent rearrangement of reactive carbenium intermediates to produce propionaldehyde, acetone, and allyl alcohol. At higher temperatures, intra-molecular addition with secondary hydroxyl group and hydrogen transfer results in the formation of by-products such as propanol and/or 2-ethyl-4-methyl-1,3-dioxolane (1,3-dioxolane), etc.

The novel solid alumina catalyst described herein exhibits superior performance for 1,2-propanediol dehydration compared to other typical alumina samples (e.g., commercial CATALOX SBA-200 γ-alumina, nano-powder γ-alumina, and PUROLAX BTa-240 η-alumina). As shown in Table 2 below, under identical reaction conditions and catalyst loading weight, the site time yield was about 1.5 kg_{reactant}kg⁻¹_{catalyst}hr⁻¹ on the novel solid alumina composition, which is ˜35% higher than that on the other commercial alumina catalysts (0.75-1.1 kg_{reactant}kg⁻¹_{catalyst}hr⁻¹). This significant improvement indicates that at mild reaction condition (250° C.), the novel solid alumina composition is more active, which thus enable a more cost and energy-efficient process.

Besides improved activity, the novel solid alumina composition also exhibited higher selectivity to the primary dehydration products with lower selectivity to undesirable byproducts. Propionaldehyde was identified as the primary product on the novel solid alumina composition, with a 64% selectivity. Side products including 2-ethyl-4-methyle-1,3-dioxolane and propanol were also observed, but with relatively low selectivity of 11%, and 15%, respectively. Other the novel solid alumina composition catalysts, however, exhibited significantly lower selectivity to the primary dehydration product propionaldehyde, which were 33% on PUROLAX BTa-240 η-alumina and CATALOX SBA-200 γ-alumina, and 43% on nano-powder γ-alumina even at lower conversion. On the other hand, a much higher selectivity toward the undesired 2-ethyl-4-methyle-1,3-dioxolane was observed on other commercial alumina catalysts, ranging from 23-28%. As 2-ethyl-4-methyle-1,3-dioxolane is preferentially formed on acid sites with weak strength, the suppression of ethyl-4-methyle-1,3-dioxolane formation on the novel solid alumina composition was likely due to its stronger acid strength.

TABLE 2
Dehydration of 1,2-propanediol over the novel solid alumina composition,
as compared to various commercial alumina catalysts

| | | PUROLAX | CATALOX | |
|---|---|---|---|---|
| | Novel solid | BTa-240 η- | SBA-200 | nano-powder |
| | alumina | alumina | γ-alumina | γ-alumina |
| | composition | (Sasol) | (Sasol) | (Thermofisher) |
| Conversion (%) | 31 ± 3 | 19 ± 3 | 15 ± 1 | 22 ± 1 |
| Site time yield | 1.5 ± 0.2 | 1.0 ± 0.2 | 0.75 ± 0.05 | 1.1 ± 0.1 |
| (kg_{reactant}kg⁻¹_{catalyst}hr⁻¹) | | | | |
| Selectivity (mol %) | | | | |
| Propionaldehyde | 64 ± 9 | 33 ± 2 | 33 ± 3 | 29 ± 3 |
| 2-Ethyl-4-methyle-1,3- | 11 ± 3 | 26 ± 9 | 23 ± 2 | 28 ± 5 |
| Dioxolane | | | | |
| Propanol | 15 ± 5 | 8 ± 3 | 11 ± 2 | 15 ± 2 |
| Allyl alcohol | 1.5 ± 0.3 | 0.6 ± 0.4 | 1.3 ± 0.2 | 1.0 ± 0.3 |
| Others | 9 ± 3 | 32 ± 7 | 32 ± 1.2 | 28 ± 5 |

Reaction conditions: 2 hour reaction time, 250° C., 150 mg catalyst, 300 rpm rotation rate

### Glycerol Dehydration:

Catalytic testing for glycerol dehydration was performed in a 50 mL batch reactor (Parr instruments 4590 micro stirred reactor system). In a typical experiment, the reactor was loaded with the catalyst (0.15 g) and glycerol (2.5 g) dissolved in a solvent consisting of 22.5 mL of water. The reactor was sealed, purged with nitrogen at ambient temperature. The reactor was then heated to 220° C. and once the desired temperature was attained, usually within 30 minutes, the stirring speed was set to 500 rpm, signifying the start of reaction. Following the fixed-time batch run for 2 hours, the heating and stirring were simultaneously stopped. The reactor was allowed to cool in air, after which product samples were taken out and a known amount of internal standard (1,4-butanediol) was added. The product samples were firstly centrifuged using VWR fixed angle centrifuge at 4000 RPM for 10 minutes to separate the catalyst particles from the liquid product samples. Then the centrifuged liquid samples were filtered through a polyethersulfone syringe membrane with 0.2 μm pore size to further separate the fine particles remaining in the liquid samples. These samples were analyzed by an Agilent Technologies 8890 gas chromatograph equipped with a flame ionization detector (FID). All the samples were injected automatically by an Agilent Technologies 7693 Series auto-injector with a 10 μL syringe. A DB-WAX Ultra inert capillary column (30 m×0.25 mm I.D.×0.50 μm film thickness) was used for separation of different species. Two experiments were performed under identical experiment conditions to calculate the average and standard deviation. We closed the carbon mass balance at about 98-105% for this preliminary test.

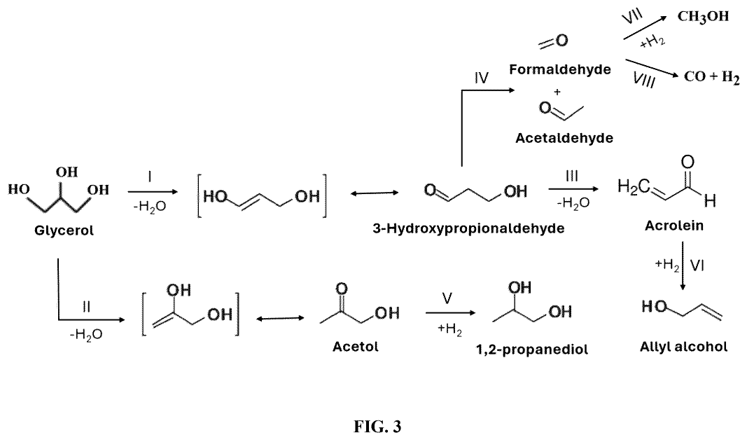

Dehydration of glycerol (FIG. 3) is initiated either by the central —OH (Step I) or terminal —OH (Step II), which results in parallel formation of two enol intermediates. The enols undergo rapid rearrangement to 3-hydroxypropionaldehyde (3-HPA) or acetol, respectively. Among these, 3-HPA, which is often assumed as the reactive intermediate in acrolein synthesis particularly under high-temperature conditions where aldol condensation reaction of acetaldehyde with formaldehyde occurs. This intermediate readily undergoes further dehydration for the production of the desirable acrolein (Step III). Subsequent hydrogenation of acrolein results in the formation of allyl alcohol (Step VI). The unstable intermediate 3-HPA would also decompose according to a reversed aldol condensation (Step IV), to acetaldehyde and formaldehyde, and a follow up hydrogenation or decomposition of formaldehyde would result in t the formation of methanol or CO and H2 (steps VII and VIII). Additionally, hydrogenation of the carbonyl group in acetol (Step V) leads to the formation of 1,2 propanediol.

The novel solid alumina catalyst exhibits superior performance for glycerol dehydration compared to other typical alumina samples, including commercial CATALOX SBA-200 γ-alumina, nano-powder γ-alumina, and PURALOX BTa-240 η-alumina. As shown in table 3, under identical reaction conditions and catalyst loading weight, the site time yield is about 0.09 kg_{reactant}kg⁻¹_{catalyst}hr⁻¹ on BCM-1 η-alumina catalyst, which is ˜50% higher than that on other catalysts (0.05-0.07 kg_{reactant}kg⁻¹_{catalyst}hr⁻¹). This significant improvement indicates that, even at mild reaction condition (220° C.), the novel alumina catalyst herein is more active in polyol dehydration applications, which thus may enable a more cost and energy-efficient process in the future.

TABLE 3
Dehydration of glycerol over novel solid alumina composition
as compared to various commercial alumina catalysts.

| | | | CATALOX SBA- | nano- |
|---|---|---|---|---|
| | BCM-1 | PUROLAX BTa-240 | 200 | powder |
| | η-alumina | η-alumina | γ-alumina | γ-alumina |
| | (this work) | (Sasol) | (Sasol) | (Thermofisher) |
| Conversion (%) | 1.13 ± 0.49 | 0.62 ± 0.21 | 0.81 ± 0.18 | 0.77 ± 0.15 |
| Site time yield | 0.09 ± 0.04 | 0.05 ± 0.02 | 0.07 ± 0.01 | 0.06 ± 0.01 |
| (kg_{reactant}kg⁻¹_{catalyst}hr⁻¹) | | | | |
| Selectivity (mol %) | | | | |
| 1,2-propanediol | 58.2 ± 1.7 | 20.7 ± 13.7 | 24.2 ± 19 | 0.33 ± 0.5 |
| Acetol | 11.1 ± 0.9 | 29.6 ± 4.2 | 19.7 ± 3.4 | 27.7 ± 5.9 |
| Acrolein | 13.6 ± 2 | 22.5 ± 8.8 | 35.5 ± 13.1 | 46.8 ± 0.06 |
| Allyl alcohol | 5.6 ± 2.5 | 10.9 ± 3.2 | 9.3 ± 2 | 9.7 ± 1.8 |
| Propionaldehyde | 2.8 ± 0.4 | 9.2 ± 4.5 | 6.6 ± 1.1 | 12.2 ± 7.8 |
| 2-Ethyl-4-methyl- | 1.9 ± 1.5 | 5.8 ± 2.6 | 4.1 ± 0.4 | 3 ± 0.45 |
| 1,3-Dioxolane | | | | |

Reaction conditions: 2 h reaction time, 220° C., 150 mg catalyst, 500 rpm rotation rate

In sum, compared to the commercial γ-alumina and η-alumina, the novel solid alumina composition described herein was more active production polyol dehydration reactions. The superior performance can bring in significant improvement on energy and cost of polyol dehydration process.

### Embodiments for Catalysts

1. A solid alumina composition prepared from an acid-treated precursor composition, said precursor composition comprising an alumina hydroxide (Al(OH)₃), aluminum oxyhydroxide (AlO(OH)), or a mixture thereof;
   wherein the solid alumina composition comprises η-alumina characterized by having an X-ray powder diffraction pattern comprising the peaks at 19.6±0.5 °2θ (e.g., 19.6±0.2 °2θ) and 66.8±0.5 °2θ (e.g., 66.8±0.2 °2θ); and
   wherein the solid alumina composition has an acidity measured by an NH₃-TPD test, characterized by:
   (1) an acid site density ranging from about 200 to 800 μmol/g (such as from about 250 to about 800, from about 300 to about 800, or from about 400 to about 800 μmol/g); and/or
   (2) an acid strength distribution of:
   about 20%-70% weak acid sites (such as about 30-50%, about 30-40%, or about 35-40%),
   about 30%-80% medium acid sites (such as about 50-70%, about 60-70%, or about 60-65%), and
   about 0-20% strong acid sites (such as about 0-10%, about 0-5%, or about 0-2%).
2. The solid alumina composition of catalyst embodiment 1, which comprises η-alumina at a high purity, characterized by a ratio of a peak intensity at 19.6±0.5 °2θ (e.g., 19.6±0.2 °2θ), relative to a peak intensity at 66.8±0.5 °2θ (e.g., 66.8±0.2 °2θ), being about 3% or greater, preferably from about 3% to about 10%, or preferably from about 6% to about 10%.
3. The solid alumina composition of catalyst embodiment 1, which has an acidity measured by an NH₃-TPD test, characterized by:
   (1) an acid site density ranging from about 200 to 800 μmol.
4. The solid alumina composition of catalyst embodiment 1, which has an acidity measured by an NH₃-TPD test, characterized by:
   (2) an acid strength distribution of:
   about 20%-70% weak acid sites (such as about 30-50%, about 30-40%, or about 35-40%),
   about 30%-80% medium acid sites (such as about 50-70%, about 60-70%, or about 60-65%), and
   about 0-20% strong acid sites (such as about 0-10%, about 0-5%, or about 0-2%).
5. The solid alumina composition of catalyst embodiment 1, which comprises an acidity measured by an NH₃-TPD test, characterized by:
   (1) an acid site density ranging from about 250 to about 800, preferably from about 300 to about 800, or preferably from about 400 to about 800 μmol/g.
6. The solid alumina composition of catalyst embodiment 1, wherein the aluminum hydroxide comprises bayerite.
7. The solid alumina composition of catalyst embodiment 1, wherein the aluminum oxyhydroxide comprises boehmite (γ-AlO(OH)), pseudo-boehmite (e.g., finely crystalline boehmite), or a mixture thereof.
8. The solid alumina composition of catalyst embodiment 1, wherein the precursor composition comprises a mixture of bayerite and boehmite or pseudo-boehmite, optionally containing over 50 wt % (such as over 60 wt %, over 70 wt %, over 80 wt %, over 90 wt %, or over 95 wt %) bayerite.
9. The solid alumina composition of catalyst embodiment 1, wherein an acid to obtain the acid-treated precursor composition, comprises an acid selected from the group consisting of an inorganic acid, a halogen acid, an organic acid, and a mixture thereof.
10. The solid alumina composition of catalyst embodiment 9, wherein the acid comprises an inorganic acid selected from the group consisting of sulfuric acid, boric acid, nitric acid, and a mixture thereof.
11. The solid alumina composition of catalyst embodiment 9, wherein the acid comprises a halogen acid selected from the group consisting of hydrochloric acid, hydrobromic acid, and a mixture thereof.
12. The solid alumina composition of catalyst embodiment 9, wherein the acid comprises an organic acid selected from the group consisting of acetic acid, citric acid, malic acid, tartaric acid, propionic acid, butyric acid, valeric acid, lactic acid, hydracrylic acid, glyceric acid, levulinic acid, malonic acid, glutaric acid, succinic acid, pimelic acid, and a mixture thereof.
13. The solid alumina composition of catalyst embodiment 9, wherein the acid comprises an acid selected from the group consisting of nitric acid, acetic acid, citric acid, malic acid, tartaric acid, or a mixture thereof.
14. A process for preparing a solid alumina composition, the process comprising:
   treating a precursor composition comprising an alumina hydroxide (Al(OH)₃), aluminum oxyhydroxide (AlO(OH)), or a mixture thereof with an acid, to form an acid-treated precursor composition;
   dispersing the acid-treated precursor composition into a homogenous aqueous suspension of particles;
   drying the homogenous aqueous suspension of particles; and
   calcinating the dried particles, to form a solid alumina composition.
15. The process of catalyst embodiment 14, wherein the dispersing is carried out by sonication, stirring, shaking, or a combination thereof.
16. The process of catalyst embodiment 14, further comprising:
   adding a surfactant to the homogenous aqueous suspension of particles.
17. The process of catalyst embodiment 16, wherein the surfactant comprises a cationic surfactant, an anionic surfactant, or a combination thereof.
18. The process of catalyst embodiment 17, wherein the cationic surfactant comprises a salt of a quaternary ammonium cation selected from the group consisting of cetyltrimethylammonium, cetylpyridinium, polydially dimethyl ammonium, and a combination thereof, and
   wherein the anionic surfactant comprises a surfactant selected from the group consisting of an alkyl sulfate, an alkyl-ether sulfate, an alkylbenzene sulfonate, a perfluoroalkanesulfonate, an alkyl-aryl ether phosphate, an alkyl ether phosphate, sodium stearate, and a combination thereof.
19. The process of catalyst embodiment 17, wherein the non-ionic surfactant comprises at least one member selected from the group consisting of a fatty alcohol ethoxylate, an alkyl phenol ethoxylate, a fatty acid alkoxylate, an ethylene oxide/propylene oxide copolymer, and a poly(ethylene oxide)-based surfactant.
20. The process of catalyst embodiment 19, wherein the poly(ethylene oxide)-based surfactant comprises at least one of TERGITOL 15-S-5, TERGITOL 15-S-7, TERGITOL 15-S-9, and TERGITOL 15-S-12.
21. The process of catalyst embodiment 14, wherein the surfactant comprises cetyltrimethylammonium bromide, poly-diallyldimethylammonium chloride, or Tergitol 15-S-7.
22. The process of catalyst embodiment 14, wherein the drying is carried out at a temperature ranging from about 20° C. to about 200° C., preferably from about 50° C. to about 150° C., or preferably from about 90° C. to about 100° C.
23. The process of catalyst embodiment 14, further comprising, prior to calcinating:
   cooling the dried particles to an ambient temperature.
24. The process of catalyst embodiment 14, wherein the calcinating is carried out at a temperature ranging from about 400° C. to about 750° C., preferably from about 500° C. to about 650° C.
25. The process of catalyst embodiment 14, wherein the calcinating is carried out at a ramp rate of about 0.5° C./minute to about 2° C./minute (such as about 1° C./minute), from ambient temperature to reach a final temperature of from about 500° C. to about 650° C. (such as from about 600° C. to about 650° C.), and optionally maintaining at the final temperature for about 4-12 hours (e.g., about 6-10 hours, about 8-10 hours, or about 10 hours).
26. The process of catalyst embodiment 17, wherein the calcinating is carried out in a stepwise manner, comprising:
   a first calcinating from ambient temperature to a reach a first temperature ranging from about 400° C. to about 450° C. (such as about 400° C.), and maintaining at the first temperature for about 1-3 hours (e.g., about 2 hours);
   a second calcinating from the first temperature to a second temperature ranging from about 450° C. to about 550° C. (such as about 500° C.), and maintaining at the second temperature for about 1-3 hours (e.g., about 2 hours); and
   a third calcinating from the second temperature to the final temperature, and maintaining at the final temperature for about 4-8 hours (e.g., about 6 hours).
27. A solid alumina composition prepared according to the process of catalyst embodiment 14, wherein the solid alumina composition comprises η-alumina characterized by having an X-ray power diffraction pattern comprising the peaks at 19.6.0±0.5 and 66.8±0.5 °2θ.
28. The solid alumina composition according to catalyst embodiment 27, wherein the solid alumina composition is an acid type, optionally having an acidity measured by a NH₃-TPD test characterized by:
   (1) an acid site density ranging from about 200 to 800 μmol/g (such as from about 250 to about 800, from about 300 to about 800, or from about 400 to about 800 μmol/g); and/or
   (2) an acid strength distribution of:
      about 20%-70% weak acid sites (such as about 30-50%, about 30-40%, or about 35-40%),
      about 30%-80% medium acid sites (such as about 50-70%, about 60-70%, or about 60-65%), and
      about 0-20% strong acid sites (such as about 0-10%, about 0-5%, or about 0-2%).
29. The solid alumina composition of catalyst embodiment 27, wherein the solid alumina composition comprises η-alumina at a high purity, characterized by the ratio of the peak intensity at 20.0 (±0.2)° 2θ relative to the peak intensity at 67.0 (±0.2)° 2θ being about 3% or greater (such as ranging from about 3% to about 10%, or from about 6% to about 10%).

### Polyol Dehydration Embodiments

1. A process for a polyol dehydration, comprising:
   providing a feedstock containing a polyol, and
   dehydrating the polyol in the presence of a solid alumina composition as a catalyst or catalyst support for the dehydration reaction to obtain a dehydration product;
   wherein the solid alumina composition is prepared from a precursor composition comprising an alumina hydroxide (Al(OH)₃), aluminum oxyhydroxide (AlO(OH)), or a mixture thereof.
2. The process of Polyol Dehydration Embodiment 1, wherein the feedstock is a bio-based feedstock.
3. The process of Polyol Dehydration Embodiment 1, wherein the polyol is glycerol, ethane diol (i.e., ethylene glycol), a propane diol (such as 1,2-propanediol (i.e., propylene glycol), or 1,3-propanediol), or a butanediol (such as 1,2-butanediol, 1,3-butanediol, or 1,4-butanediol).
4. The process of Polyol Dehydration Embodiment 1, wherein the polyol is derived from sugar (e.g., maltitol, sorbitol, xylitol, erythritol, or isomalt), plant oil (e.g., castor oil or cottonseed oil), cellulose, or hemicellulose.
5. The process of any one of the proceeding Polyol Dehydration Embodiments, wherein the dehydration reaction has an improved reaction rate, characterized by a higher site time yield (i.e., a turnover rate) under a mild reaction conditions (e.g., at a reaction temperature ranging from about 200° C. to about 300° C., from about 220° C. to about 270° C., or at about 250° C.), wherein the site time yield is at least 10% (such as at least 15%, at least 20%, at least 25%, at least 30%, at least 35%, at least 40%, at least 45%, at least 50%, at least 60%, at least 70%, at least 80%, at least 90%, or at least 100%) higher, compared to the site time yield of a dehydration reaction carried out under the same conditions but in the presence of a commercial η- or γ-alumina as the catalyst or catalyst support.
6. The process of Polyol Dehydration Embodiment 5, wherein the site time yield of the dehydration reaction is at least 35% higher, at least 50% higher, or at least 100% higher, compared to the site time yield of a dehydration reaction carried out under the same conditions but in the presence of a commercial η- or γ-alumina as the catalyst or catalyst support.
7. The process of Polyol Dehydration Embodiment 1, wherein:
   the polyol is 1,2-propanediol, and
   the dehydration reaction has a site time yield ranging from about 1.3 to about 1.7 kg_{reactant}kg⁻¹_{catalyst}hr⁻¹, under mild reaction conditions (e.g., at a reaction temperature ranging from about 200° C. to about 300° C., from about 220° C. to about 270° C., or at about 250° C.).
8. The process of any one of Polyol Dehydration Embodiments 1-4, wherein the dehydration reaction has an improved selectivity toward a primary dehydration product, formed from eliminating a water molecule from the polyol, wherein the selectivity is improved by at least 50% (such as at least 60%, at least 70%, at least 80%, at least 90%, or at least 100%), compared to a dehydration reaction carried out under the same conditions but in the presence of a commercial η- or γ-alumina as the catalyst or catalyst support.
9. The process of any one of Polyol Dehydration Embodiments 1-4, wherein the dehydration product contains a reduced amount of byproduct, wherein the byproduct is reduced by at least 50% (such as at least 60%), compared to a dehydration reaction carried out under the same conditions but in the presence of a commercial η- or γ-alumina as the catalyst or catalyst support.
9a. The process of Polyol Dehydration Embodiment 9, wherein the dehydration product is formed from a dehydration process that involves eliminating a molecule other than water molecule, hydrogen transfer, and/or intra-molecular addition with secondary hydroxy group.
10. The process of Polyol Dehydration Embodiment 8, wherein the polyol is glycerol, and the primary dehydration product comprises acrolein.
11. The process of Polyol Dehydration Embodiment 8, wherein the polyol is 1,2-propanediol, and the primary dehydration product comprises propionaldehyde.
12. The process of Polyol Dehydration Embodiment 11, wherein the dehydration product has:
   a selectivity toward propionaldehyde improved by about 50% (such as about 60%, about 70%, about 80%, or about 90%) or higher, and
   a reduced amount of 2-ethyl-4-methyle-1,3-dioxolane, which is reduced by about 50% (or about 60%) or more, compared to a dehydration reaction process carried out under the same conditions but in the presence of a commercial η- or γ-alumina as the catalyst or catalyst support.
13. The process of Polyol Dehydration Embodiment 11, wherein the dehydration reaction is carried out at a weight ratio of catalyst:polyol of 0.05-0.15 (e.g., about 0.1), a reaction temperature ranging from about 200° C. to about 300° C. (e.g., from about 220° C. to about 270° C., or at about 250° C.), and/or a stirring speed ranging from 250-350 rpm (e.g., about 300 rpm).
14. The process of Polyol Dehydration Embodiment 13, wherein the dehydration product contains:
   greater than about 60 mol % propionaldehyde,
   no more than about 11 mol % 2-ethyl-4-methyle-1,3-dioxolane, and/or
   no more than about 15 mol % propanol.
15. The process of any one of Polyol Dehydration Embodiments 1-14, wherein:
   the solid alumina composition comprises η-alumina characterized by having an X-ray powder diffraction pattern comprising the peaks at 19.6±0.5 °2θ (e.g., 19.6±0.2 °2θ) and 66.8±0.5 °2θ (e.g., 66.8±0.2 °2θ); and
   the solid alumina composition has an acidity measured by an NH₃-TPD test, characterized by:
   (1) an acid site density ranging from about 200 to 800 μmol/g (such as from about 250 to about 800, from about 300 to about 800, or from about 400 to about 800 μmol/g); and/or
   (2) an acid strength distribution of:
      a. about 20%-70% weak acid sites (such as about 30-50%, about 30-40%, or about 35-40%),
      b. about 30%-80% medium acid sites (such as about 50-70%, about 60-70%, or about 60-65%), and
      c. about 0-20% strong acid sites (such as about 0-10%, about 0-5%, or about 0-2%).
16. The process of Polyol Dehydration Embodiment 15, wherein the solid alumina composition comprises η-alumina at a high purity, characterized by a ratio of a peak intensity at 19.6±0.5 °2θ (e.g., 19.6±0.2 °2θ), relative to a peak intensity at 66.8±0.5 °2θ (e.g., 66.8±0.2 °2θ), being about 3% or greater, preferably from about 3% to about 10%, or preferably from about 6% to about 10%.
17. The process of Polyol Dehydration Embodiment 15, wherein the aluminum hydroxide comprises bayerite.
18. The process of Polyol Dehydration Embodiment 15, wherein the aluminum oxyhydroxide comprises boehmite (γ-AlO(OH)), pseudo-boehmite (e.g., finely crystalline boehmite), or a mixture thereof.
19. The process of Polyol Dehydration Embodiment 15, wherein the precursor composition comprises a mixture of bayerite and boehmite or pseudo-boehmite, optionally containing over 50 wt % (such as over 60 wt %, over 70 wt %, over 80 wt %, over 90 wt %, or over 95 wt %) bayerite.
20. The process of Polyol Dehydration Embodiment 15, wherein an acid to obtain the acid-treated precursor composition comprises an acid selected from the group consisting of an inorganic acid (e.g., sulfuric acid, boric acid, nitric acid, or a mixture thereof), a halogen acid (e.g., hydrochloric acid, hydrobromic acid, or a mixture thereof), an organic acid (e.g., acetic acid, citric acid, malic acid, tartaric acid, propionic acid, butyric acid, valeric acid, lactic acid, hydracrylic acid, glyceric acid, levulinic acid, malonic acid, glutaric acid, succinic acid, pimelic acid, or a mixture thereof), and a mixture thereof.
21. The process of Polyol Dehydration Embodiment 20, wherein the acid comprises an acid selected from the group consisting of nitric acid, acetic acid, citric acid, malic acid, tartaric acid, and a mixture thereof.
22. The process of any one of Polyol Dehydration Embodiments 1-14, wherein the solid alumina composition is prepared by a process comprising:
   treating a precursor composition comprising an alumina hydroxide (Al(OH)₃), aluminum oxyhydroxide (AlO(OH)), or a mixture thereof with an acid, to form an acid-treated precursor composition;
   dispersing the acid-treated precursor composition into a homogenous aqueous suspension of particles;
   drying the homogenous aqueous suspension of particles (e.g., at a temperature ranging from about 20° C. to about 200° C., preferably from about 50° C. to about 150° C., or preferably from about 90° C. to about 100° C.); and
   calcinating the dried particles (e.g., at a temperature ranging from about 400° C. to about 750° C., preferably from about 500° C. to about 650° C.), to form a solid alumina composition.
23. The process of Polyol Dehydration Embodiment 22, wherein the dispersing is carried out by sonication, stirring, shaking, or a combination thereof.
24. The process of Polyol Dehydration Embodiment 22, wherein the process for preparing the solid alumina composition further comprises:
   adding a surfactant (e.g., a cationic surfactant, an anionic surfactant, or a combination thereof) to the homogenous aqueous suspension of particles; and/or
   prior to calcinating, cooling the dried particles to an ambient temperature.
25. The process of Polyol Dehydration Embodiment 22, wherein the calcinating is carried out at a ramp rate of about 0.5° C./minute to about 2° C./minute (such as about 1° C./minute), from ambient temperature to reach a final temperature of from about 500° C. to about 650° C. (such as from about 600° C. to about 650° C.), and optionally maintaining at the final temperature for about 4-12 hours (e.g., about 6-10 hours, about 8-10 hours, or about 10 hours).
26. The process of Polyol Dehydration Embodiment 22, wherein the calcinating is carried out in a stepwise manner, comprising:
   a first calcinating from ambient temperature to a reach a first temperature ranging from about 400° C. to about 450° C. (such as about 400° C.), and maintaining at the first temperature for about 1-3 hours (e.g., about 2 hours);
   a second calcinating from the first temperature to a second temperature ranging from about 450° C. to about 550° C. (such as about 500° C.), and maintaining at the second temperature for about 1-3 hours (e.g., about 2 hours); and
   a third calcinating from the second temperature to the final temperature, and maintaining at the final temperature for about 4-8 hours (e.g., about 6 hours).

1. A process for a polyol dehydration, comprising:
providing a feedstock containing a polyol, and
dehydrating, via a dehydration reaction, the polyol in the presence of a solid alumina composition as a catalyst or catalyst support, to obtain a dehydration product;
wherein the solid alumina composition is prepared from a precursor composition comprising an alumina hydroxide (Al(OH)₃), aluminum oxyhydroxide (AlO(OH)), or a mixture thereof.

2. (canceled)

3. The process of claim 1, wherein the polyol is glycerol, ethane diol, a propane diol, or a butanediol.

4. The process of claim 1, wherein the polyol comprises glycerol, 1,2-propanediol, or a combination thereof.

5. The process of claim 1,
wherein the dehydration reaction has an improved reaction rate, characterized by a higher site time yield under a mild reaction conditions, and
wherein the site time yield is at least 10% higher, compared to the site time yield of a dehydration reaction carried out under the same conditions but in the presence of a commercial η- or γ-alumina as the catalyst or catalyst support.

6. The process of claim 5, wherein the site time yield of the dehydration reaction is at least 35% higher, compared to the site time yield of a dehydration reaction carried out under the same conditions but in the presence of a commercial η- or γ-alumina as the catalyst or catalyst support.

7. The process of claim 1, wherein:
the polyol is 1,2-propanediol, and
the dehydration reaction has a site time yield ranging from about 1.3 to about 1.7 kg_{reactant}kg⁻¹_{catalyst}hr⁻¹, under mild reaction conditions.

8. The process of claim 1, wherein the dehydration reaction has an improved selectivity toward a primary dehydration product, formed from eliminating a water molecule from the polyol, wherein the selectivity is improved by at least 50%, compared to a dehydration reaction carried out under the same conditions but in the presence of a commercial η- or γ-alumina as the catalyst or catalyst support.

9. The process of claim 1, wherein the dehydration product comprises a reduced amount of byproduct, wherein the byproduct is reduced by at least 50%, compared to a dehydration reaction carried out under the same conditions but in the presence of a commercial η- or γ-alumina as the catalyst or catalyst support.

10. The process of claim 9, wherein the dehydration product is formed from a dehydration process that involves eliminating a molecule other than water molecule, hydrogen transfer, and/or intra-molecular addition with secondary hydroxy group.

11. The process of claim 8, wherein the polyol is glycerol, and the primary dehydration product comprises acrolein.

12. The process of claim 8, wherein the polyol is 1,2-propanediol, and the primary dehydration product comprises propionaldehyde.

13. The process of claim 12, wherein the dehydration product has:
a selectivity toward propionaldehyde improved by about 50% or higher, and
a reduced amount of 2-ethyl-4-methyle-1,3-dioxolane, which is reduced by about 50% or more, compared to a dehydration reaction process carried out under the same conditions but in the presence of a commercial η- or γ-alumina as the catalyst or catalyst support.

14. The process of claim 12, wherein the dehydration reaction is carried out at a weight ratio of catalyst:polyol of 0.05-0.15, a reaction temperature ranging from about 200° C. to about 300° C., and/or a stirring speed ranging from 250-350 rpm.

15. The process of claim 14, wherein the dehydration product comprises:
greater than about 60 mol % propionaldehyde,
no more than about 11 mol % 2-ethyl-4-methyle-1,3-dioxolane, and/or
no more than about 15 mol % propanol.

16. The process of claim 1, wherein:
the solid alumina composition comprises η-alumina characterized by having an X-ray powder diffraction pattern comprising the peaks at 19.6±0.5 °2θ and 66.8±0.5 °2θ; and
the solid alumina composition has an acidity measured by an NH₃-TPD test, characterized by:
(1) an acid site density ranging from about 200 to 800 μmol/g; and/or
(2) an acid strength distribution of:
a. about 20%-70% weak acid sites,
b. about 30%-80% medium acid sites, and
c. about 0-20% strong acid sites.

17. The process of claim 16, wherein the solid alumina composition comprises η-alumina at a high purity, characterized by a ratio of a peak intensity at 19.6±0.5 °2θ, relative to a peak intensity at 66.8±0.5 °2θ, being about 3% or greater.

18. The process of claim 17, wherein the alumina hydroxide comprises bayerite.

19. The process of claim 17, wherein the aluminum oxyhydroxide comprises boehmite (γ-AlO(OH)), pseudo-boehmite (finely crystalline boehmite), or a mixture thereof.

20. The process of claim 17, wherein the precursor composition comprises a mixture of bayerite and either boehmite or pseudo-boehmite.

21-22. (canceled)

23. The process of claim 1, wherein the solid alumina composition is prepared by a process comprising:
treating a precursor composition comprising an alumina hydroxide (Al(OH)₃), aluminum oxyhydroxide (AlO(OH)), or a mixture thereof with an acid, to form an acid-treated precursor composition;
dispersing the acid-treated precursor composition into a homogenous aqueous suspension of particles;
drying the homogenous aqueous suspension of particles; and
calcinating the dried particles,
to form a solid alumina composition.

24-27. (canceled)
